(19) United States
(12) Patent Application Publication
(10) Pub. No.: US 2017/0189580 A1
(43) Pub. Date: Jul. 6, 2017

(54) CARBON NANOTUBE-BASED FIBERS, USES THEREOF AND PROCESS FOR MAKING SAME

(71) Applicants: Stefania POLIZU, Montreal (CA); Philippe POULIN, Talence (FR); Oumarou SAVADOGO, Montreal (CA); L'Hocine YAHIA, Montreal (CA)

(72) Inventors: Stefania POLIZU, Montreal (CA); Philippe POULIN, Talence (FR); Oumarou SAVADOGO, Montreal (CA); L'Hocine YAHIA, Montreal (CA)

(21) Appl. No.: 15/404,017

(22) Filed: Jan. 11, 2017

Related U.S. Application Data
(63) Continuation of application No. 12/233,336, filed on Sep. 18, 2008, now abandoned.
(60) Provisional application No. 60/960,153, filed on Sep. 18, 2007.

(51) Int. Cl. A61L 27/44; A61L 27/58

(52) U.S. Cl. CPC A61L 27/443; A61L 27/58; A61L 2400/12; A61L 2430/32

(57) ABSTRACT

A biocompatible and biodegradable carbon nanotube-based fiber capable of stimulating and sustaining cell proliferation and stimulating and sustaining nerve regeneration is disclosed herein. The biocompatible and biodegradable carbon nanotube-based fiber comprising at least one carbon nanotube; a biodegradable copolymer; and a coagulating polymer. The present disclosure also relates to a process fro producing such a fiber.

### CROSS REFERENCE TO RELATED APPLICATIONS

The present application is a continuation of U.S. application Ser. No. 12/233,336, filed Sep. 18, 2008, which claims the benefit of U.S. Provisional Application No. 60/960,153 filed Sep. 18, 2007. The entire contents of the above-referenced application are incorporated into the present application by reference.

### FIELD

The present specification relates to carbon nanotube-based fibers, uses thereof and a process for making same. More specifically, but not exclusively, the present specification relates to carbon nanotube-based fibers capable of stimulating and sustaining cell proliferation, uses thereof, as well as a process for making same. The present specification also relates to carbon nanotube-based fibers suitable as biomaterials.

### BACKGROUND

Carbon nanotubes (CNTs) typically comprise single graphite sheets rolled up into a seamless cylinder (tube). Carbon nanotubes can be synthesized by arc-discharge, laser ablation or chemical vapor deposition methods, involving the use of various electrodes, supports or catalysts. The cylindrical structure can be made of a single layer of carbon atoms, single-wall nanotube (SWNT), or multiple layers of carbon atoms, multi-wall carbon nanotube (MWNT), (FIG. 1 and FIG. 2) [1]. Carbon nanotubes typically possess a diameter of a few nanometers and a length ranging from about one nanometer to about several microns. The carbon nanotube wall comprises an extended sp² hybridized carbon network, which reduces the carbon nanotube reactivity and solubility in water.

Carbon nanotubes constitute attractive biomaterials because of their vast specific area, outstanding aspect ratio (i.e. length to diameter ratio), excellent elastic modulus, good electric conductivity, and good capacity of activation. In the last few years, formulations of carbon nanotube-based macroscopic materials have been reported in various forms of composites, assemblies, arrays and hybrid systems [2, 3, 4, 5, 6].

Haddon's and Webster's research groups have shown carbon nanotubes to be useful biomaterials for the proliferation of cells. Haddon reported on the application of carbon nanotubes for neural research. Indeed, Haddon described carbon nanotubes as supports for nerve cell growth and as substrates for probes with neuronal function, at the nanometer scale [7]. Multi-wall nanotubes of diameters that matched those of nerve fibers (ranging from about 10 nm to about 100 nm) were used to illustrate that embryonic rat-brain neurons could be grown thereon. Furthermore, Haddon demonstrated that carbon nanotubes coated with bioactive molecules, including 4-hydroxynonenal, could stimulate neurite growth with extensive branching.

Webster defined the role of nanosized morphology for neural cells [8, 9] and illustrated the efficiency of carbon nanofibers (CNF) as neural biomaterials. The in vitro cytocompatibility of carbon nanofibers [8] revealed the importance of the fibrous characteristic to diminish the astrocyte function, reducing glial scar tissue formation.

It was also proposed that carbon nanofibers may contribute to the restoration of a damaged neuronal circuit [10, 11, 12, 13]. The ability of carbon nanotubes and carbon nanofibers to regenerate bone cells have also been investigated [14, 15, 16, 17, 18].

Biodegradable biomaterials containing carbon nanotubes are desirable to enhance the growth of regenerative cells. The control of the biodegradability provides for the manipulation of the system's biofunctionality. However, carbon nanotubes are typically not biodegradable. Therefore, the release of carbon nanotubes as nanosized particles in biological systems may potentially be undesirable. A partially biodegradable biomaterial containing a carbon nanotube network could prevent the release of carbon nanotubes while preserving their arrangement in a macroscopic material.

Most neural implants are made of silicon-based materials and induce glial scar tissue formation, which is a recurrent problem in the field of neural prosthetics. More recently, polylactic-co-glycolic acid (PLGA) was reported as a material that could serve as a neural guide and that could alleviate scar tissue formation [19]. Polylactic-co-glycolic acid comprises a randomly sequenced copolymer of polylactic acid (PLA) and polyglycolic acid (PGA), the copolymer being a biodegradable and non-toxic material, allowing for medical and pharmaceutical applications (e.g.: support for sutures, fracture fixation devices, and drug delivery systems).

Biodegradation of polylactic-co-glycolic acid occurs via hydrolysis. The rate of degradation can be modulated by using different monomer ratios and varying their molecular weight, viscosity, conformation and structure end (i.e. capped versus non-capped ends). Polylactic acid is more hydrophobic than polyglycolic acid and will influence the hydrophilic character of the copolymer. The carbonyl functional groups of both polylactic acid and polyglycolic acid have the capability of hydrogen bonding.

A challenge for the elaboration of carbon nanotube-based biomaterials (i.e. materials containing carbon nanotubes) is to integrate the nanoscale characteristics of carbon nanotubes into macroscopic equivalents: carbon nanotube arrays, carbon nanotube composites and carbon nanotube hybrid materials. For biological or medical applications, carbon nanotubes are usually chemically modified and combined with various biopolymers and/or biological molecules, via covalent bonding, to yield a biomaterial that has very specific characteristics. Carbon nanotube-based biomaterials usually comprise carefully selected components exhibiting one or more desired distinctive features.

The assembly of carbon nanotube-based materials may be performed by micro and nanofabrication methods including thermal processes such as extrusion or injection molding, electro-spinning, dry spinning and wet spinning. [2, 3, 4, 5, 6]. Particle coagulation spinning (PCS) involves a wet spinning process which enables the incorporation of carbon nanotubes into a polymeric coagulating agent. [20, 21] Particle coagulation spinning provides macroscopically aligned carbon nanotubes, which enhances the responsiveness of carbon nanotubes when in contact with living cells.

Particle coagulation spinning entails the injection of a carbon nanotube aqueous dispersion into a rotating bath containing a solution of coagulating polymer (e.g. polyvinyl alcohol (PVA)) (FIG. 2). The alignment of the carbon nanotubes is induced by the fluid direction and velocity. The fiber is formed as soon as the pre-fiber agglomeration is contacted with the coagulating polymer. The stream of the rotating bath carries the fiber away from the injection port, ensuring the formation of a string-like fiber. Long ribbons may be formed with a diameter ranging from a few micrometers to 100 μm. The produced carbon nanotube-based fiber displays flexibility, high resistance to torsion and is plastic-like at room temperature. Particle coagulation spinning is generally free from chemical reactions, providing for low levels of contamination, which is essential for biomaterial applications.

The present specification refers to a number of documents, the content of which is herein incorporated by reference in their entirety.

### SUMMARY

The present specification broadly relates to novel carbon nanotube-based fibers capable of stimulating and sustaining cell proliferation and uses thereof. The carbon nanotube-based fibers comprise at least one carbon nanotube, a biodegradable copolymer and a coagulating polymer. The present specification also relates to a process for making a carbon nanotube-based fiber capable of stimulating and sustaining cell proliferation.

In an embodiment, the present specification relates to a biocompatible carbon nanotube-based fiber capable of stimulating and sustaining cell proliferation.

In an embodiment, the present specification relates to a biocompatible carbon nanotube-based fiber capable of stimulating and sustaining nerve regeneration.

In an embodiment, the present specification relates to a biocompatible carbon nanotube-based fiber capable of stimulating and sustaining cell proliferation, the biocompatible carbon nanotube-based fiber comprising: (i) at least one single-wall carbon nanotube; (ii) a biodegradable copolymer; and (iii) a coagulating polymer.

In an embodiment, the present specification relates to a biocompatible carbon nanotube-based fiber capable of stimulating and sustaining nerve regeneration, the biocompatible carbon nanotube-based fiber comprising: (i) at least one single-wall carbon nanotube; (ii) a biodegradable copolymer; and (iii) a coagulating polymer.

In an embodiment, the present specification relates a biocompatible carbon nanotube-based fiber capable of stimulating and sustaining cell proliferation, the biocompatible carbon nanotube-based fiber comprising: (i) at least on multi-wall carbon nanotube; (ii) a biodegradable copolymer; and (iii) a coagulating polymer.

In an embodiment, the present specification relates a biocompatible carbon nanotube-based fiber capable of stimulating and sustaining nerve regeneration, the biocompatible carbon nanotube-based fiber comprising: (i) at least on multi-wall carbon nanotube; (ii) a biodegradable copolymer; and (iii) a coagulating polymer.

In an embodiment, the present specification relates to carbon nanotube-based biomaterials capable of stimulating and sustaining cell proliferation.

In an embodiment, the present specification relates to carbon nanotube-based biomaterials capable of stimulating and sustaining nerve regeneration.

In an embodiment, the present specification relates to a carbon nanotube-based biomaterial capable of stimulating and sustaining cell proliferation, the carbon nanotube-based biomaterial comprising: (i) at least one single-wall carbon nanotube; (ii) a biodegradable copolymer; and (iii) a coagulating polymer.

In an embodiment, the present specification relates to a carbon nanotube-based biomaterial capable of stimulating and sustaining nerve regeneration, the carbon nanotube-based biomaterial comprising: (i) at least one single-wall carbon nanotube; (ii) a biodegradable copolymer; and (iii) a coagulating polymer.

In an embodiment, the present specification relates to a carbon nanotube-based biomaterial capable of stimulating and sustaining cell proliferation, the carbon nanotube-based biomaterial comprising: (i) at least one multi-wall carbon nanotube; (ii) a biodegradable copolymer; and (iii) a coagulating polymer.

In an embodiment, the present specification relates a carbon nanotube-based biomaterial capable of stimulating and sustaining nerve regeneration, the carbon nanotube-based biomaterial comprising: (i) at least one multi-wall carbon nanotube; (ii) a biodegradable copolymer; and (iii) a coagulating polymer.

In an embodiment, the present specification relates to a process for producing a carbon nanotube-based fiber, the process comprising: (i) providing an aqueous carbon nanotube dispersion; (ii) providing an aqueous biodegradable copolymer suspension; (iii) mixing the aqueous carbon nanotube dispersion with the aqueous copolymer suspension to provide a colloidal mixture; and (iv) contacting the colloidal mixture with a coagulating polymer.

The foregoing and other objects, advantages and features of the present specification will become more apparent upon reading of the following non restrictive description of illustrative embodiments thereof, given by way of example only with reference to the accompanying drawings, and which should not be interpreted as limiting the scope of the present specification.

### BRIEF DESCRIPTION OF THE DRAWINGS

In the appended drawings:

FIG. 1 (which is labeled “Prior Art”) is a drawing of single-wall nanotubes (SWNT);

FIG. 2 (which is labeled “Prior Art”) is a drawing of multi-wall carbon nanotubes (MWNT);

FIG. 3 (which is labeled “Prior Art”) is an illustration of a particle coagulation spinning apparatus, showing the injection of a carbon nanotube aqueous dispersion into a rotating bath containing a solution of coagulating polymer (e.g. polyvinyl alcohol (PVA));

FIG. 4 shows a Scanning Electron Microscopy (SEM) micrograph of a carbon nanotube-based fiber, in accordance with an embodiment of the present specification;

FIG. 5 shows a Scanning Electron Microscopy (SEM) micrograph illustrating the presence of fibrils in a carbon nanotube-based fiber which was fractured in liquid nitrogen (the presence of fibrils contribute to the mesoscale architecture of single-wall carbon nanotube-based fibers), in accordance with an embodiment of the present specification;

FIG. 6 shows an Atomic Force Microscopy (AFM) image illustrating a topographic view of a single-wall carbon nanotube-based fiber, in accordance with an embodiment of the present specification;

FIG. 7 shows an Atomic Force Microscopy (AFM) 3D-image illustrating a topographic view of a multi-wall carbon nanotube-based fiber displaying oriented periodic bundles, in accordance with an embodiment of the present specification;

FIG. 8 shows a Scanning Electron Microscopy (SEM) micrograph illustrating the organization and orientation of single-wall carbon nanotubes in relation to nanoparticles of RG 502, in accordance with an embodiment of the present specification;

FIG. 9 shows a Scanning Electron Microscopy (SEM) micrograph illustrating the organization and orientation of single-wall carbon nanotubes in relation to nanoparticles of RG 503H, in accordance with an embodiment of the present specification;

FIG. 10 shows a Scanning Electron Microscopy (SEM) micrograph illustrating the organization and orientation of multi-wall carbon nanotubes in relation to nanoparticles of RG 502, in accordance with an embodiment of the present specification;

FIG. 11 is a graph illustrating the loss tangent (Tan δ) as a function of temperature for single-wall carbon nanotube-based fibers of different bulk composition, in accordance with an embodiment of the present specification;

FIG. 12 is a graph illustrating the weight loss in function of temperature for single-wall carbon nanotube-based fibers of different bulk composition, as obtained by Thermogravimetric Analysis (TGA), in accordance with an embodiment of the present specification;

FIG. 13 is a graph illustrating the storage modulus (G′) and loss modulus (G″) as a function of temperature for a single-wall carbon nanotube-based fiber, as obtained by Dynamic Mechanical Analysis (DMA), in accordance with an embodiment of the present specification;

FIG. 14 is a graph illustrating the stress-strain evolution for single-wall and multi-wall carbon nanotube-based fibers, in accordance with an embodiment of the present specification;

FIG. 15 shows a Scanning Electron Microscopy (SEM) micrograph illustrating a carbon nanotube-based fiber following a two (2) week incubation period in a Phosphate Buffered Saline (PBS) solution, in accordance with an embodiment of the present specification, the carbon nanotubes emerging from the fiber surface showing biodegradation;

FIG. 16 shows a Scanning Electron Microscopy (SEM) micrograph illustrating a carbon nanotube-based fiber following a three (3) week incubation period in a Phosphate Buffered Saline (PBS) solution, in accordance with an embodiment of the present specification, the carbon nanotubes emerging from the fiber surface showing surface erosion and more advanced biodegradation;

FIG. 17 is a histogram illustrating the variation of optical density values of PC12 cells cultured using different single-wall carbon nanotube-based fibers, in accordance with an embodiment of the present specification;

FIG. 18 is a histogram illustrating the variation of optical density values of PC12 cells cultured using different multi-wall carbon nanotube-based fibers, in accordance with an embodiment of the present specification;

FIG. 19 shows a microscopic image (×100 magnification) of PC12 cells cultured on MWR3-2 fibers, following a two (2) day culture period, illustrating a strong spatial correlation between cells and fiber, in accordance with an embodiment of the present specification;

FIG. 20 shows a microscopic image (×100 magnification) of PC12 cells cultured on collagen treated SWR2-2 fibers, following a two (2) day culture period, illustrating cells spreading in the fiber's direction in addition to their adhesion thereto, in accordance with an embodiment of the present specification;

FIG. 21 shows a Scanning Electron Microscopy (SEM) micrograph illustrating PC12 cells cultured on SWR3-1 fibers (on a collagen coated glass slide in the absence of Neural Growth Factors (NGFs)), illustrating cells spreading in the fiber's direction in addition to their adhesion thereto, in accordance with an embodiment of the present specification;

FIG. 22 shows a depth view of the PC12 cells of FIG. 19, illustrating the morphological characteristics of the PC 12 cells and the formation of neurites following a three (3) day culture period;

FIG. 23 shows a microscopic image (×100 magnification) of PC12 cells cultured on MWR3-1 fibers, following a two (2) day culture period in the presence of Neural Growth Factors (NGFs) and in the absence of collagen, illustrating their adhesion and alignment alongside the MWR3-1 fibers, in accordance with an embodiment of the present specification;

FIG. 24 shows a microscopic image (×100 magnification) of PC12 cells cultured on collagen treated SWR3-1 fibers, following a three (3) day culture period in the presence of Neural Growth Factors (NGFs), illustrating the formation of neurites on the SWR3-1 fibers and in the fiber's surrounding, in accordance with an embodiment of the present specification;

FIG. 25 shows a microscopic image (×100 magnification) of PC12 cells cultured on SWR3-1 fibers, illustrating cell adhesion, alignment and neurite extension, in accordance with an embodiment of the present specification;

FIG. 26 shows a fluorescence microscopic image (×100 magnification) of PC12 cells cultured on MWR2-1 fibers, following a three (3) day culture period, illustrating cell adhesion, alignment and neurite extension, in accordance with an embodiment of the present specification;

FIG. 27 shows a fluorescence microscopic image (×100 magnification) of PC12 cells cultured on collagen treated MWR2-1 fibers, in the absence of Neural Growth Factors (NGFs), illustrating cell adhesion, alignment and neurite extension, in accordance with an embodiment of the present specification;

FIG. 28 shows a microscopic image (×100 magnification) of PC12 cells cultured on SWR3-1 fibers, illustrating cell adhesion and growth in the absence collagen, in accordance with an embodiment of the present specification;

FIG. 29 shows a microscopic image (×100 magnification) of PC12 cells cultured on SWR3-1 fibers, following a two (2) day culture period (slide 1 showing the cells being seeded onto the fibers; slide 2 showing cell migration along the fibers; and slide 3 showing cell migration following the two (2) day culture period) in accordance with an embodiment of the present specification;

FIG. 30 shows a microscopic image (×100 magnification) of human skin fibroblasts cultured on MWR2-2 fibers, following a two (2) day culture period, illustrating cell adhesion and growth in an elongated shape, in accordance with an embodiment of the present specification;

FIG. 31 shows a microscopic image (×100 magnification) of human skin fibroblast cultured on MWR2-2 fibers, following a three (3) day culture period, illustrating cell organization, in accordance with an embodiment of the present specification;

FIG. 32 shows a fluorescence microscopic image (×100 magnification) of human skin fibroblast cultured on MWR2-2 fibers, following a three (3) day culture period, illustrating cells spreading along the fibers, in accordance with an embodiment of the present specification; and

FIG. 33 shows a microscopic image (×100 magnification) of human skin fibroblasts cultured on SWR3-1 fibers, following a two (2) day culture period (slide 1 showing the cells being seeded onto the fibers; slide 2 showing cell migration along the fibers; and slide 3 showing cell migration following the two (2) day culture period) in accordance with an embodiment of the present specification.

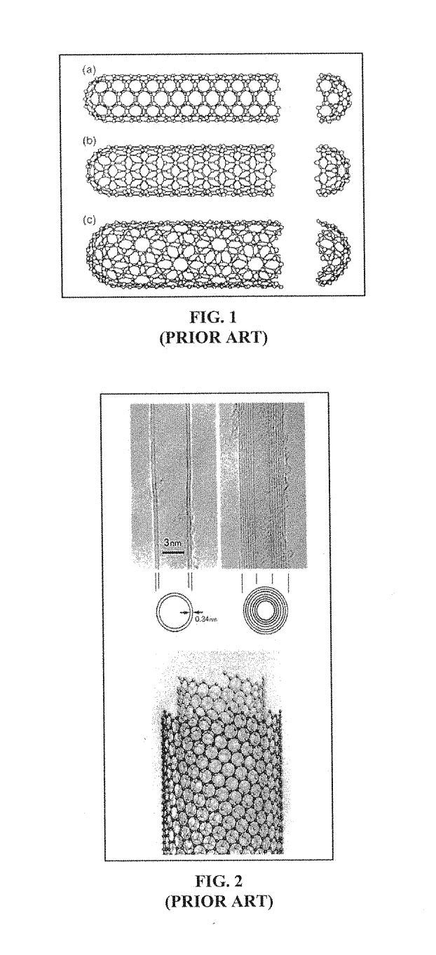

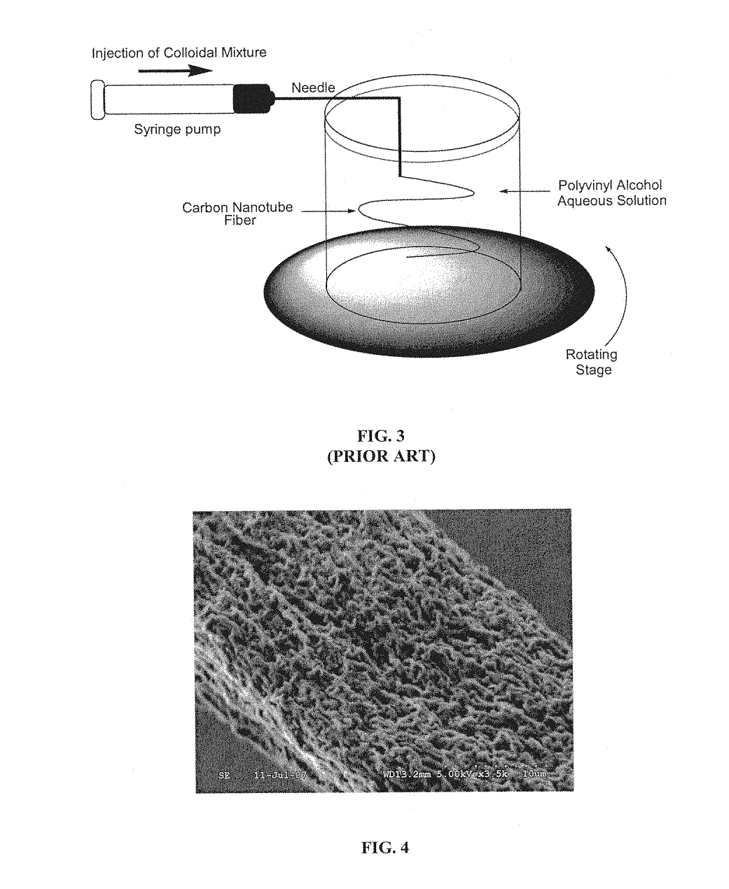

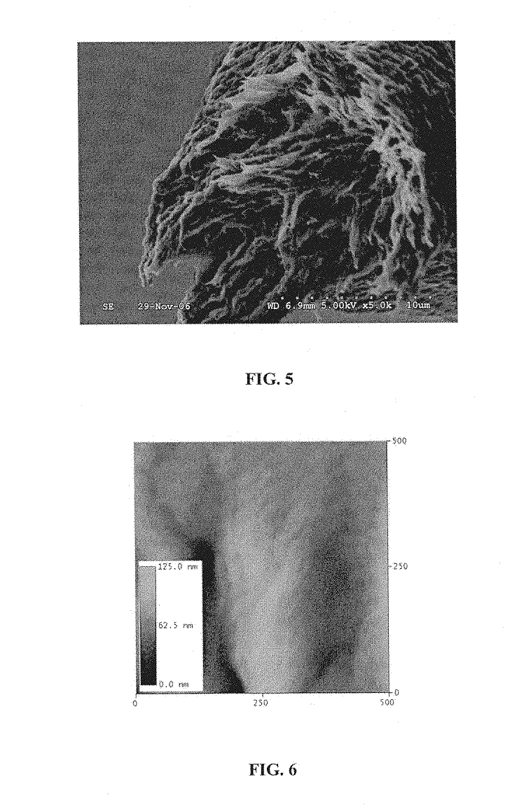

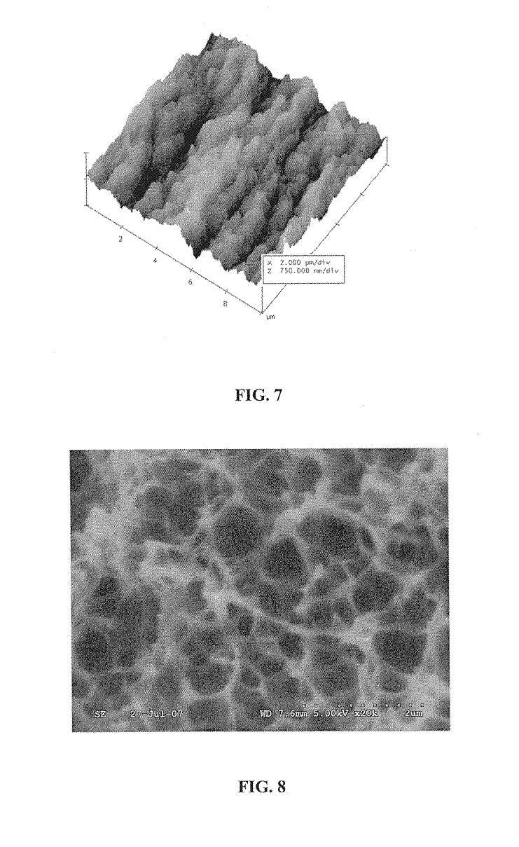

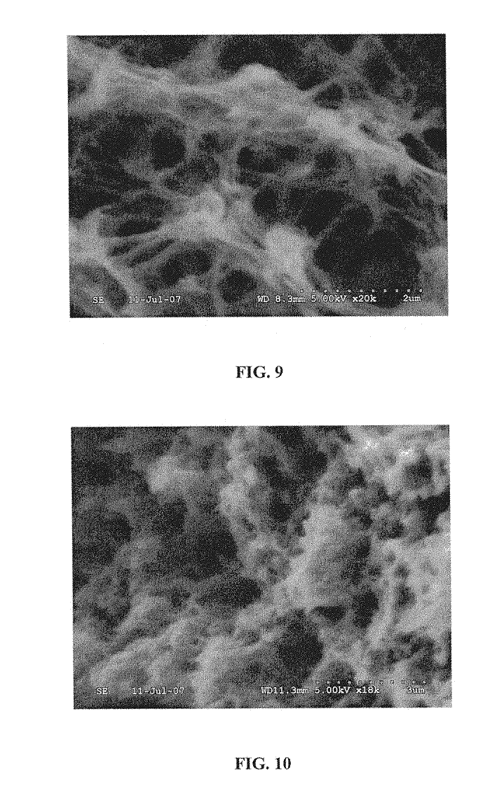

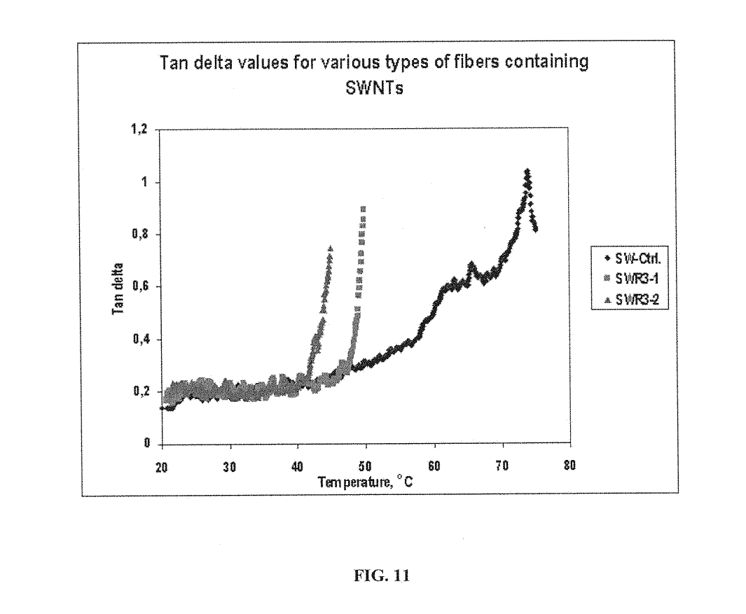

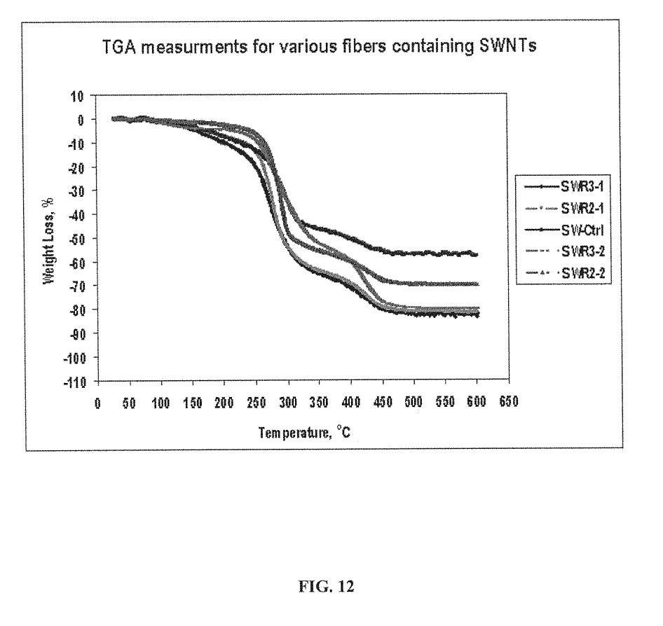

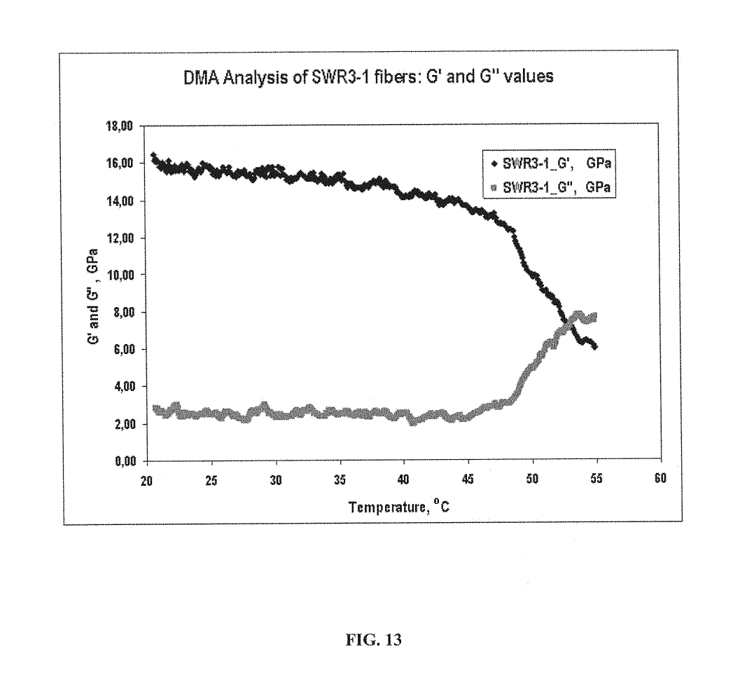

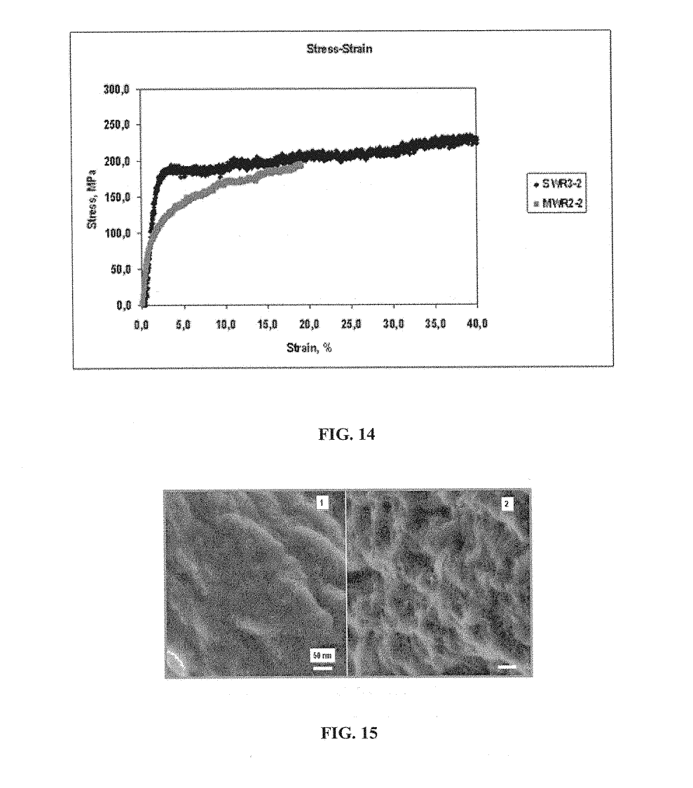

### DETAILED DESCRIPTION

In order to provide a clear and consistent understanding of the terms used in the present specification, a number of definitions are provided below. Moreover, unless defined otherwise, all technical and scientific terms as used herein have the same meaning as commonly understood to one of ordinary skill in the art to which this specification pertains.

The use of the word “a” or “an” when used in conjunction with the term “comprising” in the claims and/or the specification may mean “one”, but it is also consistent with the meaning of “one or more”, “at least one”, and “one or more than one”. Similarly, the word “another” may mean at least a second or more.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “about” is used to indicate that a value includes an inherent variation of error for the device or the method being employed to determine the value.

As used in this specification, the following abbreviations have the following denotations: SWNT: Single-Wall Carbon Nanotube; MWNT: Multi-Wall Carbon Nanotube; SDS: Sodium Dodecyl Sulphate; PLGA: Polylactic-co-Glycolic Acid; R2: PLGA Resomer RG502; R3: PLGA Resomer RG503H; PVA: polyvinyl alcohol; NGF: Neural Growth Factor; SEM: Scanning Electron Microscopy; AFM: Atomic Force Microscopy; DMA: Dynamic Mechanical Analysis; TGA: Thermogravimetric Analysis; CVD: Chemical Vapor Deposition; LV-SEM: Low-Vacuum Scanning Electron Microscopy; and PBS: Phosphate Buffered Saline.

The present specification broadly relates to carbon nanotube-based fibers capable of stimulating and sustaining cell proliferation, the carbon nanotube-based fibers comprising: (i) at least one carbon nanotube; (ii) a biodegradable copolymer; and (iii) a coagulating polymer. The present specification also broadly relates to carbon nanotube-based fibers capable of stimulating and sustaining nerve regeneration, the carbon nanotube-based fibers comprising: (i) at least one carbon nanotube; (ii) a biodegradable copolymer; and (iii) a coagulating polymer.

In a non-limiting embodiment of the present specification, the carbon nanotube-based fibers are assembled in non-covalent fashion, relying on hydrogen-bonding and Van der Waals interactions, thus preserving the carbon nanotubes' inertness. In a further non-limiting embodiment of the present specification, the chemical composition of the carbon nanotube-based fibers is controlled to modulate its physical and chemical properties.

In a non-limiting embodiment of the present specification, particle coagulation spinning is used to produce a homogenous three-component hybrid biomaterial. In a further non-limiting embodiment of the present specification, the biomaterial comprises a fibrous nanoscale architecture which is prone to interactions with living cells.

In an embodiment, the present specification relates to a process for producing a carbon nanotube-based fiber, the process comprising: (i) providing an aqueous carbon nanotube dispersion; (ii) providing an aqueous biodegradable copolymer suspension; (iii) mixing the aqueous carbon nanotube dispersion with the aqueous copolymer suspension to provide a colloidal mixture; and (iv) contacting the colloidal mixture with a coagulating polymer. In a non-limiting embodiment of the present specification, the aqueous media further comprises additives. Non-limiting examples of suitable additives include antibodies, chemical entities, collagen, drugs, growth factors, laminine, oligonucleotides, peptides, peptide derivatives, siRNA and mixtures thereof. In an embodiment of the present specification, the aqueous carbon nanotube dispersion comprises a surfactant. A non-limiting example of a suitable surfactant includes sodium dodecyl sulphate (SDS). A homogenous and stable dispersion of carbon nanotubes can be produced in an aqueous solution of sodium dodecyl sulphate.

In an embodiment of the present specification, an aqueous copolymer suspension is obtained by first dissolving a copolymer in an organic solvent. Non-limiting examples of suitable copolymers include polylactic-co-glycolic acid, polylactide-block-polyethylene oxide, polylactide-co-polycaprolactone, ethylene-co-vinyl alcohol and mixture thereof. Non-limiting examples of suitable organic solvents include acetone, dichloromethane, and mixture thereof. In light of the present specification, it is believed to be within the capacity of a skilled technician to determine and select other suitable copolymers and solvents. In a non-limiting embodiment of the present specification, the concentration of copolymer in organic solvent ranges from about 15 mg/mL to about 50 mg/mL. Water is subsequently added to the copolymer solution to produce a suspension comprising copolymer nanoparticles. The particle size of the nanoparticles is dictated by the ratio of organic solution to water volume. The organic solvent is subsequently removed from the copolymer suspension.

In a non-limiting embodiment of the present specification, an aqueous polylactic-co-glycolic acid suspension is produced. In a non-limiting embodiment of the present specification, the concentration of polylactic-co-glycolic acid in organic solvent ranges from about 15 mg/mL to about 50 mg/mL. Water is subsequently added to the polylactic-co-glycolic acid solution to produce a suspension comprising polylactic-co-glycolic acid nanoparticles. The volume ratio of organic solution to water typically ranges from about 40:60 to about 60:40. The nanoparticles typically comprise a diameter ranging from about 100 nm to about 300 nm. The organic solvent is subsequently removed from the polylactic-co-glycolic acid suspension.

In a non-limiting embodiment of the present specification, the organic solvent is evaporated by constant mechanical stirring at room temperature. In light of the present disclosure, it is believed to be within the capacity of a skilled technician to determine and select other suitable methods to remove the organic solvent.

The carbon nanotube dispersion and the copolymer suspension may be mixed in different ratios to obtain a colloidal mixture of rod-like and spherical particles.

In the colloidal mixture, polylactic-co-glycolic acid particles maximize the separation between the carbon nanotubes, preventing their aggregation. The efficient alignment of the carbon nanotubes improves the electrical activity of the carbon nanotube-based fiber.

In an embodiment of the present specification, the colloidal mixture may be introduced in a syringe and injected into a co-flowing stream of coagulation polymer solution. Non-limiting examples of suitable coagulating polymers include polyvinyl alcohol, carboxymethyl cellulose, sodium alginate, hyaluronic acid and mixture thereof. In light of the present disclosure, it is believed to be within the capacity of a skilled technician to determine and select other suitable coagulating polymers. The syringe may be placed on a syringe pump in order to better control the speed of the injection. Injection of the colloidal mixture induces a rearrangement of the carbon nanotubes and polylactic-co-glycolic acid particles into a pre-fiber type agglomeration. A fiber is formed as soon as the pre-fiber agglomeration makes contact with the coagulating polymer.

The ratio of carbon nanotube dispersion to copolymer suspension (e.g. polylactic-co-glycolic acid suspension) can be optimized to obtain a fiber having a desired texture, morphology, hydrophilic/hydrophobic balance, biocompatibility, biodegradability, mechanical behavior and orientation. Moreover, the copolymer polylactic-co-glycolic acid contributes to the elasticity and strain deformation of the fiber, which affect the substrate-cell interactions. The presence of hydrophobic copolymer reduces the propensity of the fiber to swell when immersed into a biological medium, thus providing an elastic and flexible support for the cells.

A fraction of polyvinyl alcohol forms a stable network with polylactic-co-glycolic acid via hydrogen bonding. A further fraction of coagulating polymer (e.g. polyvinyl alcohol) replaces the surfactant molecules (e.g. sodium dodecyl sulphate) on the surface of the carbon nanotubes, neutralizing the effect of sodium dodecyl sulphate. Hydrogen bonding typically occurs between the polyvinyl alcohol hydroxyl groups (—OH) and the polylactic-co-glycolic acid carbonyl (—C═O) and carboxyl (—COOH) groups.

Molecular weight, concentration and the degree of hydrolysis of polyvinyl alcohol control the viscosity of the coagulation bath, modulating the elasticity of the fiber. The concentration of polyvinyl alcohol dictates the type of interaction the host polymer exhibits with the pre-fiber. The concentration of polyvinyl alcohol solution typically ranges from about 2% to about 10% in water, thus allowing for polyvinyl alcohol to act as both surfactant and coagulating agent. In an embodiment of the present specification, the polyvinyl alcohol has a molecular weight ranging from about 100 KDa to about 200 KDa. Polyvinyl alcohol comprises a copolymer of polyvinyl acetate and polyvinyl alcohol; the degree of hydrolysis being indicative of the percentage of hydroxyl groups. In an embodiment of the present specification, the polyvinyl alcohol comprises a degree of hydrolysis of about 89%, which corresponds to a copolymer comprising about 89% polyvinyl alcohol and about 11% polyvinyl acetate.

In an embodiment of the present specification, the carbon nanotube-based fibers are separated from the solution of polyvinyl alcohol and introduced in a rinsing bath. Excess polyvinyl alcohol is removed from the fiber by washing with distilled water. A sufficient amount of polyvinyl alcohol remains on the fibers in order to maintain the carbon nanotubes and polylactic-co-glycolic acid network. Finally, the fiber is suspended and dried under ambient conditions.

As confirmed by experimental characterization of the carbon nanotube fibers, the carbon nanotube-based fibers are typically organized on three different levels: (i) macroscopic scale: thread/cylinder like-shapes, which surfaces are characterized by roughness, oriented column, oriented fibers, periodicity, spherical contours; (ii) mesoscopic scale: presence of fibrous ramifications, oriented fibrils; and (iii) nanoscopic scale: individual nanotubes and bundles aligned parallel to the axis, spherical nanoparticles and their assemblies.

In an embodiment of the present specification, the carbon nanotube-based fiber exhibits biocompatibility and bioactivity tailored by the experimental parameters of fabrication. In a further embodiment of the present specification, the carbon nanotube-based fiber is biodegradable.

Excess polyvinyl alcohol is removed from the fiber by washing with distilled water, thus exposing more of the biodegradable component (e.g. polylactic-co-glycolic acid). More rinsing operations will thus result in the exposure of more biodegradable component. The exposure of the biomaterial can thus be further controlled by the number of rinsing operations In the course of the biodegradation process, a new space is created on the fibrous structure, promoting cell growth on the porous surface while the carbon nanotube network provides a guide.

In an embodiment of the present specification, the composition of the carbon nanotube-based fiber ranges from about 15% to about 50% of carbon nanotubes and from about 50% to about 85% of polymer as illustrated in Table 1.

TABLE 1
Description and Composition of the Synthesized
Carbon Nanotube-Based Fibers.

| | | Fiber Composition | |
|---|---|---|---|
| Fiber | Fiber Description | Polymer (%) | CNT (%) |
| SW-Ctrl. | SWNT and PVA | 57.8 | 42.2 |
| | (control) | | |
| SWR2-1 | SWNT, RG 502 and PVA | 81.5 | 18.5 |
| | (low PLGA concentration) | | |
| SWR3-1 | SWNT, RG 503H and PVA | 81.5 | 18.5 |
| | (low PLGA concentration) | | |
| SWR2-2 | SWNT, RG 502 and PVA | 70.0 | 30.0 |
| | (high PLGA concentration) | | |
| SWR3-2 | SWNT, RG 503H and PVA | 80.4 | 19.6 |
| | (high PLGA concentration) | | |
| MW-Ctrl. | MWNT and PVA (control) | 58.7 | 41.3 |
| MWR2-1 | MWNT, RG 502 and PVA | 62.0 | 38.0 |
| | (low PLGA concentration) | | |
| MWR3-1 | MWNT, RG 503H and PVA | 69.5 | 30.5 |
| | (low PLGA concentration) | | |
| MWR2-2 | MWNT, RG 502 and PVA | 53.5 | 46.5 |
| | (high PLGA concentration) | | |
| MWR3-2 | MWNT, RG 503H and PVA | 61.5 | 38.5 |
| | (high PLGA concentration) | | |

The synthesized carbon nanotube-based fibers were characterized and a number of their mechanical and viscoelastic characteristics are illustrated hereinbelow in Table 2.

TABLE 2
Mechanical and Viscoelastic Characteristics of the Synthesized
Carbon Nanotube-based fibers.

| | | | Elastic | Storage | Less | Loss |
|---|---|---|---|---|---|---|
| Fibers | Stress | Strain | modulus | modulus | modulus | tangent |
| Name | (σ) MPa | (ε) % | (E) GPa | (G’), GPa | (G”) GPa | Tan δ |
| SW-Ctrl. | 100 | 2.5 | 6.0 | 5.8-3.2 | 0.9-3.2 | 0.12-0.73 |
| SWR2-1 | 140 | 2.0 | 11.5 | 13.2-6.5 | 2.0-6.5 | 0.15-0.81 |
| SWR3-1 | 150 | 2.5 | 18.0 | 16.0-7.4 | 2.4-7.4 | 0.18-0.90 |
| SWR2-2 | 130 | 1.9 | 11.0 | 12.0-5.9 | 2.8-3.4 | 0.20-0.50 |
| SWR3-2 | 180 | 30 | 13.0 | 9.3-6.0 | 2.0-4.4 | 0.19-0.74 |
| MW-Ctrl. | 110 | 18 | 5.0 | 4.0-1.9 | 0.8-1.2 | 0.20-0.58 |
| MWR2-1 | 200 | 25 | 11.5 | 9.1-3.7 | 1.3-2.6 | 0.16-0.71 |
| MWR3-1 | 220 | 22 | 12.0 | 11.7-4.6 | 1.9-2.6 | 0.16-0.56 |
| MWR2-2 | 240 | 20 | 11.0 | 11.0-4.5 | 0.9-1.4 | 0.15-0.30 |
| MWR3-2 | 185 | 9.5 | 10.0 | 9.0-4.6 | 1.6-1.7 | 0.18-0.37 |

### Experimental

A number of examples are provided herein below, illustrating the process for making carbon nanotube-based fibers as well their use as a biomaterial.

Dispersion of Single-Wall Carbon Nanotubes (SW-HiPCO) in Sodium Dodecyl Sulphate (SDS): Dispersion D-SW

Single-wall carbon nanotube purified powder (lot PO 257, PO 272) was purchased from Carbon Nanotechnologies Inc., and synthesized by Gas-Phase Decomposition of CO (HiPCO).

To single-wall carbon nanotubes (0.03 g, 0.3 wt. %) was added sodium dodecyl sulphate (0.10 g, 1.0 wt. %) and water (9.87 mL, 98.7 wt. %). The aqueous mixture was then ultrasonicated using a sonicator horn over a period of 60-80 min at 40 KW to yield the title dispersion D-SW.

Dispersion of Multi-Wall Carbon Nanotubes (Arkema) in Sodium Dodecyl Sulphate (SDS): Dispersion D-MW.

Multi-wall carbon nanotubes (lot NTC 3056) were purchased from Arkema, and synthesized by catalyzed Chemical Vapor Deposition (CVD).

To multi-wall carbon nanotubes (0.18 g, 0.9 wt. %) was added sodium dodecyl sulphate (0.24 g, 1.2 wt. %) and water (19.58 mL, 97.9 wt. %). The aqueous mixture was then ultrasonicated using a sonicator horn over a period of 45 min at 20 KW to yield the title dispersion D-MW.

Suspension of Polylactic-Co-Glycolic Acid (PLGA) in Water: Suspension R2-1.

Resomer RG 502 (RG 502) of PLGA was purchased from Boehringer Ingelheim (Ingelheim, Germany); RG 502 comprising a 50:50 ratio of lactic:glycolic acids, viscosity of 0.16-0.24 dlg⁻¹, and MW of 12 000 Daltons.

To a solution of Resomer RG 502 (200 mg) in acetone (13 mL) was added dropwise distilled water (12 mL). The mixture was then stirred overnight at room temperature until complete evaporation of acetone to yield the title suspension R2-1.

Suspension of Polylactic-Co-Glycolic Acid (PLGA) in Water: Suspension R3-1.

Resomer RG 503H (RG503H) of PLGA was purchased from Boehringer Ingelheim (Ingelheim, Germany); RG 503H comprising a 50:50 ratio of lactic:glycolic acids, viscosity of 0.32-0.44 dlg⁻¹, and MW of 34 000 Daltons.

To a solution of Resomer RG 503H (200 mg) in acetone (13 mL) was added dropwise distilled water (12 mL). The mixture was then stirred overnight at room temperature until complete evaporation of acetone to yield the title suspension R3-1.

Suspension of Polylactic-Co-Glycolic Acid (PLGA) in Water: Suspension R2-2.

Resomer RG 502 (RG 502) of PLGA was purchased from Boehringer Ingelheim (Ingelheim, Germany); RG 502 comprising a 50:50 ratio of lactic:glycolic acids, viscosity of 0.16-0.24 dlg⁻¹, and MW of 12 000 Daltons.

To a solution of Resomer RG 502 (600 mg) in acetone (13 mL) was added dropwise distilled water (12 mL). The mixture was then stirred overnight at room temperature until complete evaporation of acetone to yield the title suspension R2-2.

Suspension of Polylactic-Co-Glycolic Acid (PLGA) in Water: Suspension R3-2.

Resomer RG 503H (RG503H) of PLGA was purchased from Boehringer Ingelheim (Ingelheim, Germany); RG 503H comprising a 50:50 ratio of lactic:glycolic acids, viscosity of 0.32-0.44 dlg⁻¹, and MW of 34 000 Daltons.

To a solution of Resomer RG 503H (600 mg) in acetone (13 mL) was added dropwise distilled water (12 mL). The mixture was then stirred overnight at room temperature until complete evaporation of acetone to yield the title suspension R3-2.

Mixture of Single-Wall Carbon Nanotubes Dispersion (D-SW) with Polylactic-Co-Glycolic Acid Suspension (R2-1): Dispersion D-SWR2-1.

To dispersion D-SW (2 mL) was added suspension R2-1 (1 mL) and the mixture was sonicated over a period of 15 min to yield a homogenized mixture of the title dispersion D-SWR2-1.

Mixture of Single-Wall Carbon Nanotubes Dispersion (D-SW) with Polylactic-Co-Glycolic Acid Suspension (R3-1): Dispersion D-SWR3-1.

To dispersion D-SW (3 mL) was added suspension R3-1 (1 mL) and the mixture was sonicated over a period of 15 min to yield a homogenized mixture of the title dispersion D-SWR3-1.

Mixture of Single-Wall Carbon Nanotubes Dispersion (D-SW) with Polylactic-Co-Glycolic Acid Suspension (R2-2): Dispersion D-SWR2-2.

To dispersion D-SW (2 mL) was added suspension R2-2 (1 mL) and the mixture was sonicated over a period of 15 min to yield a homogenized mixture of the title dispersion D-SWR2-2.

Mixture of Single-Wall Carbon Nanotubes Dispersion (D-SW) with Polylactic-Co-Glycolic Acid Suspension (R3-2): Dispersion D-SWR3-2.

To dispersion D-SW (3 mL) was added suspension R3-2 (1 mL) and the mixture was sonicated over a period of 15 min to yield a homogenized mixture of the title dispersion D-SWR3-2.

Mixture of Multi-Wall Carbon Nanotubes Dispersion (D-MW) with Polylactic-Co-Glycolic Acid Suspension (R2-1): Dispersion D-MWR2-1.

To dispersion D-MW (7 mL) was added suspension R2-1 (1 mL) and the mixture was sonicated over a period of 15 min to yield a homogenized mixture of the title dispersion D-MWR2-1.

Mixture of Multi-Wall Carbon Nanotubes Dispersion (D-MW) with Polylactic-Co-Glycolic Acid Suspension (R3-1): Dispersion D-MWR3-1.

To dispersion D-MW (5 mL) was added suspension R3-1 (1 mL) and the mixture was sonicated over a period of 15 min to yield a homogenized mixture of the title dispersion D-MWR3-1.

Mixture of Multi-Wall Carbon Nanotubes Dispersion (D-MW) with Polylactic-Co-Glycolic Acid Suspension (R2-2): Dispersion D-MWR2-2.

To dispersion D-MW (7 mL) was added suspension R2-2 (1 mL) and the mixture was sonicated over a period of 15 min to yield a homogenized mixture of the title dispersion D-MWR2-1.

Mixture of Multi-Wall Carbon Nanotubes Dispersion (D-MW) with Polylactic-Co-Glycolic Acid Suspension (R3-2): Dispersion D-MWR3-2.

To dispersion D-MW (5 mL) was added suspension R3-2 (1 mL) and the mixture was sonicated over a period of 15 min to yield a homogenized mixture of the title dispersion D-MWR3-2.

Preparation of 5% Polyvinyl Alcohol (PVA) Aqueous Solution.

Polyvinyl alcohol powder was purchased from Sigma-Aldrich (Lot 70580); polyvinyl alcohol having a hydrolysis percentage of 88-89% and MW of 150 000 Daltons.

To PVA (50 g) was added distilled water (950 mL) and the mixture was heated to 95° C. while stirring. After 45 min, the mixture was cooled to room temperature while stirring to yield a 5% PVA aqueous solution.

General Procedure for the Injection of the Dispersion into Polyvinyl Alcohol (PVA).

A PVA solution (150-200 mL) was placed in a coagulation bath (glass cylinder of 80-100 mm in diameter and 70-90 mm in height and fixed concentrically on a rotating table). A stainless steel needle was used for injecting the dispersion into the coagulation bath. This needle (0.50 mm I.D.) was bent so that the dispersion could be injected parallel to the bath surface. The point of injection of the dispersion was at a radius of 20-30 mm from the center of the cylindrical dish, about 10-20 mm under the surface of the PVA solution, and parallel to the dish bottom. The glass cylinder containing the PVA solution was rotated at 40-60 rpm. The employed rate of 0.80-0.85 mL/min for injecting the dispersion into the coagulation bath was achieved using a syringe pump. After laminar rotational flow was established, the syringe pump was activated and the dispersion (5 mL) was injected in a direction parallel to the established flow. The coagulation of the dispersion formed a continuous spiral ribbon inside the PVA solution. The ribbon was subsequently washed in water and dried in air to form carbon nanotube-based fibers.

General Procedure for Rinsing and Drying the Carbon Nanotube-Based Fibers.

The carbon nanotube-based fiber was carefully transferred from the PVA solution into a bath of distilled water and left there over a period of 30 minutes with no stirring or agitation. This procedure was repeated three to six times, each time using a fresh bath of water. The washed ribbon was then suspended and dried under ambient conditions.

Characterization of Carbon Nanotube-Based Fibers.

Scanning Electron Microscopy (SEM).

Scanning Electron Microscopy was performed using a Hitachi System, Model S-3550N. The topography and surface morphology of each carbon nanotube-based fiber was observed. More specifically, the carbon nanotube alignment and arrangement with copolymer nanoparticles was investigated.

Atomic Force Microscopy (AFM).

Atomic Force Microscopy was performed using a Nanoscope III A controller, a multimode AFM head (Digital Instrument Santa Barbara Calif., USA) using a silicon cantilever. The imaging was performed in taping mode on a fiber surface for a selected area of 500×500 nm², 5×5 μm² and 10×10 μm², respectively. The AFM amplitude, topography, and phase scans were investigated for the selected areas of each carbon nanotube-based fiber.

Thermogravimetric Analysis (TGA).

Thermogravimetric Analysis was performed using a Setaram instrument, a high-performance Modular Thermogravimetric Analyzer TGA, at a Rate of 10° C./min under argon atmosphere at a maximum temperature of 600° C. The composition of each carbon nanotube-based fiber was determined (Data illustrated in Table 1).

Dynamic Mechanical Analysis (DMA).

Dynamic Mechanical Analysis was performed using a DMA Perkin-Elmer 7E Analyzer, in tensile mode under isochronal conditions at a frequency of 1 Hz. A force of 20 mN was applied to the carbon nanotube-based fiber while the temperature scans were ramped from 20° C. to 110° C. at a rate of 5° C./min. The modulus (stiffness) and damping (energy dissipation) properties of the carbon nanotube-based fiber were investigated under oscillatory stress. The loss modulus (G″), storage modulus (G′) and loss tangent (Tan δ), as well as the ratio of G″ and G′, were determined for each carbon nanotube-based fiber (Data illustrated in Table 2).

Mechanical Behavior—Tensile Test.

The mechanical behavior of CNT-based fibers was investigated using an Instron 4301 testing machine in tensile mode. The stress (σ), strain (ε) and Young's modulus (E) were determined for each carbon nanotube-based fiber (Data illustrated in Table 2).

Biodegradation of the Carbon Nanotube-Based Fibers.

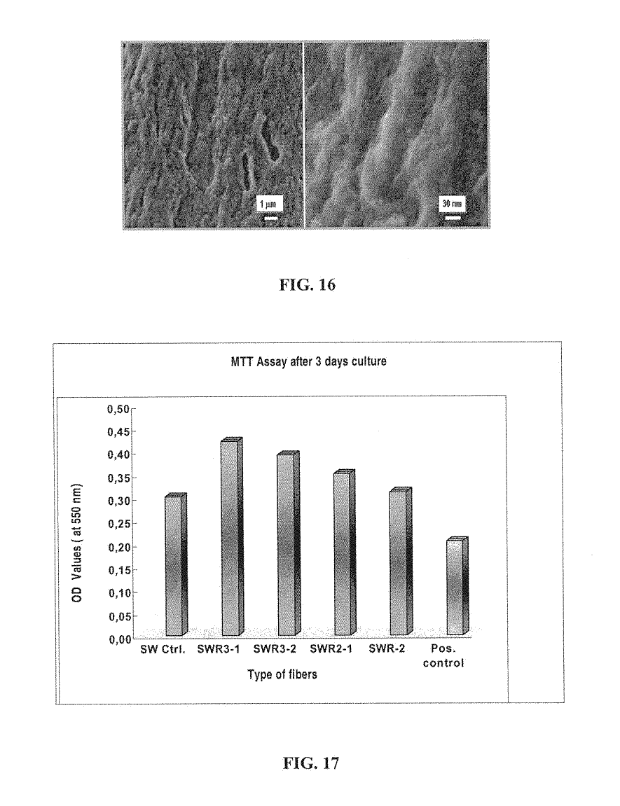

The biodegradability was assessed over a 3 week period in phosphor buffer saline (PBS), as demonstrated in FIG. 15 and FIG. 16. Nanoscale morphology and topology were investigated using Low-Vacuum Scanning Electron Microscopy (LV-SEM) equipped with an energy dispersive detector (EDS).

Biological Results.

Bio-Assays representing the capability of the carbon nanotube-based fibers of the present specification to influence the growth of living cells as well as their proliferation are provided hereinbelow.

PC12 Cells.

PC12 cells were used to investigate the ability of the carbon nanotube-based fibers of the present specification to stimulate the proliferation of nerve cells, to modulate their growth process and to contribute to the extension of neurites. These experiments demonstrate the potential use of the carbon nanotube-based fibers of the present specification as neural biomaterials for the regeneration of neural cells, particularly to facilitate nerve regeneration for injured parts of the spinal cord.

General Procedure for PC12 Cell Bio Assay.

PC12 cells, rat pheochromocytoma cells, are usually employed as a model system for neuronal development studies. Cultured in a medium containing animal blood serum, the PC12 cells adopt a round and phase bright morphology and proliferate to high density. Cultured on collagen coated plates, the adhesion to the substrate is enhanced. These cells can cease proliferating and undergo differentiation in the presence of specific trophic substances, such as neural growth factors (NGFs). The formation of neurites serves as indicator for cell differentiation. Four aspects were studied: adhesion, migration, proliferation and differentiation of the PC12 cells.

The carbon nanotube-based fibers described in Table 1 were used in the bio-assays. Pre-cleaned microscope glass slides (Erie Scientific, lot No. 2951) of 1 mm thickness were used. Rat PC12 cells, obtained from American Type Tissue Collection, were used in the cell cultures. Since rat PC12 cells are known to have poor adhesion to plastic, a collagen coating was used to improve adhesion. Type II collagen, purchased from Sigma-Aldrich Canada Ltd., was used to coat the plates and/or as solution for fiber immersion. RPMI Medium 1640, containing 10% heat-inactivated horse serum and 5% fetal bovine serum, was purchased from Gibco-BRL (Grand Island, N.Y., USA), and used as growth medium. Phosphate Buffered Saline (PBS) was purchased from Sigma-Aldrich Canada Ltd. The culture medium was supplemented with 50 ng/mL Neural Growth factors (NGF-Gibco-BRL, Grand Island, N.Y., USA) to induce cell differentiation.

General Procedure for Sample Preparation.

Microscope slides were cut into pieces (7 mm×7 mm), using a diamond cutter. The cut slides were then cleaned in an ultrasonic bath, 20 minutes in acetone, followed by 20 minutes in distilled water. The fiber sample, comprising 20 carbon nanotube-based fibers, was supported on the 7×7 mm² glass slide. The control sample was a collagen coated 7×7 mm² glass slide. The samples were placed in a 12-well tissue culture plate, which was sterilized with ethylene oxide (EO) gas at 37° C. following standard procedures, and washed once with phosphate buffered saline (PBS). For adhesion studies, the samples were placed in a 12-well tissue culture plate coated with a 0.5 mg/mL solution of sterilized Type II Collagen, and sterilized for 45 minutes.

General Procedure for MTT Assay.

The MTT test is a colorimetric metabolic assay enabling a quantification of cellular growth through changes in cell proliferation, cell viability and cytotoxicity as a response to external factors. The assay is based on the capacity of various living cell dehydrogenases to cleave MTT (3-(4,5-dimethylthiazol-2-yl)-2,5-diphenyl tetrazolium bromide) and display a dark blue formazan product. The formazan product is mostly impermeable to cell membranes, thus resulting in its accumulation within healthy cells. The process requires active mitochondria to cleave significant amounts of MTT, the yellow tetrazolium salt (MTT) is reduced to form insoluble purple formazan crystals, which are solubilized by the addition of a detergent. The number of surviving cells is directly proportional to the level of the formazan produced, allowing spectrophotometric procedures to detect changes in cell metabolism. The results were read on a multiwell scanning spectrophotometer (ELISA reader), and the relationship between cell number and absorbance was established.

After a three day culture period, the cells were transferred from the individual wells to 12 mL Sarted tubes, with 2 mL culture media. The culture media was centrifuged for 10 minutes and the supernatant was removed. The cells were then subjected to the 3-(4,5-dimethylthiazole-2-yl)-2,5-diphenyltetrazolium bromide (MTT) reduction assay. A 5 mg/mL MTT stock solution in phosphate buffered saline (PBS) was prepared. To each culture tube was added 0.2 mL of MTT stock solution followed by incubation at 37° C. After 4 h, the supernatant was removed and 0.2 mL of color development solution (0.04N HCl/isopropanol) was added to each well before continuing the incubation for an additional 15 min. The HCl converted the phenol red to yellow, which does not interfere with MTT formazan measurements. Formazan was dissolved by isopropanol and produced a homogeneous blue solution suitable for absorbance measurements. When the cells broke, the product (formazan) turns purple, turning the solution purple as well. Three samples of 200 μL of the purple solution were transferred from each well to a 96 flat-bottom well plate. The optical density was measured with the ELISA plate reader (model 680, BioRad Laboratories, Mississauga, ON, Canada) at 570 nm. The absorbance was proportional to the number of living cells present.

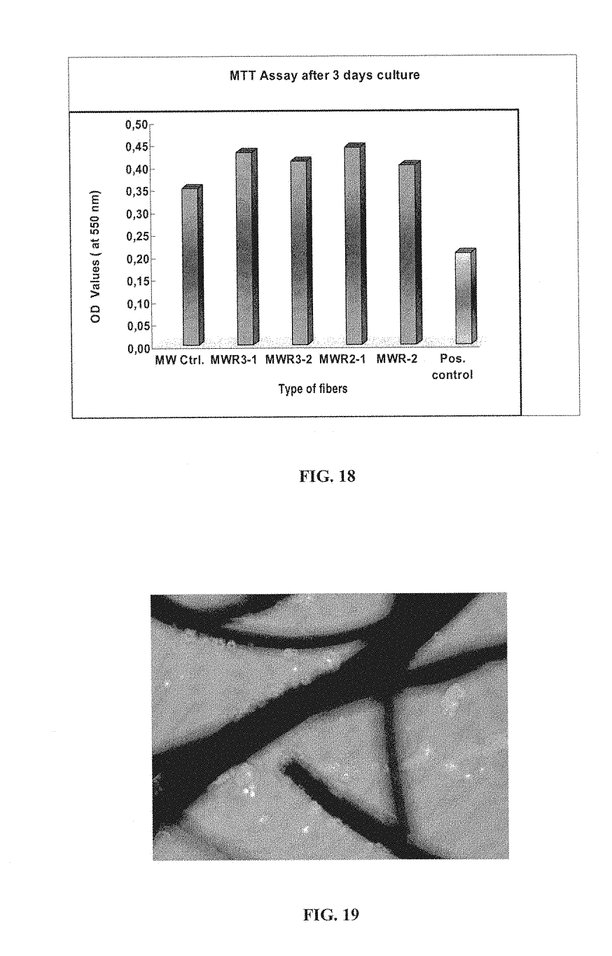

As shown in Table 3, the absorbance (Optical Density, OD) varied as a function of the carbon nanotube-based fiber. The OD values for the positive control versus the single-wall carbon nanotube-based fibers and multi-wall carbon nanotube-based fibers are represented in FIG. 17 and FIG. 18, respectively.

TABLE 3
Optical Density of the Synthesized Carbon Nanotube-
Based Fibers after MTT Assay with PC12 Cells.

| | Optical Density |
|---|---|
| Fiber | (OD) |
| Positive Control | 0.20 |
| Collagen coated plate | |
| SW-Ctrl. | 0.30 |
| SWR2-1 | 0.35 |
| SWR3-1 | 0.42 |
| SWR2-2 | 0.31 |
| SWR3-2 | 0.39 |
| MW-Ctrl. | 0.35 |
| MWR2-1 | 0.44 |
| MWR3-1 | 0.43 |
| MWR2-2 | 0.40 |
| MWR3-2 | 0.41 |

General Procedure for the Observation of Cultured PC12 Cells.

Cell Observation with Inverted Microscope.

A Nikon Eclipse E800 microscope, model Nikon, Corp., Yokohama, Japan, equipped with a digital camera for image registration was used to observe the carbon nanotube-based fibers. The carbon nanotube-based fibers were not optically transparent, preventing the visualization of cells attached to the fiber by optical microscopy. However, the images taken with the inverted microscope, at the fiber/cell interface, displayed the orientation and the presence of cells on the fibers.

Cell Observation with Scanning Electron Microscope.

The microscopic observation were performed using a Variable Pressure Scanning Electron Microscope, model S-3500N, Hitachi. After three days of culture, the samples were washed three times with phosphate buffered saline (PBS) and fixed with 4% paraformaldehyde in phosphate buffered saline (PBS) for 15 min. The fixed cells were washed with distilled water and gradually dehydrated in ethanol.

Cell Visualization by Hoechst Staining.

Hoechst staining was used to identify the cells attached to the membrane as well as to identify the formation of neurites. After three days of culture, the samples were washed three times with phosphate buffered saline (PBS) and fixed with 4% paraformaldehyde in phosphate buffered saline (PBS) for 15 min. The samples were then incubated at room temperature for 15 min in 2 mL of 2 μg/mL Hoechst solution (Hoechst 33342/PBS). The observation of stained cells was performed with an epifluorescence microscope (model Axiophot, Zeiss, Oberkochen, Germany). A qualitative analysis of the florescence images demonstrated cell growth, neurite formation and confirmed the contribution of fibrous substrate to cell adhesion.

General Procedure for PC12 Cell Adhesion and Growth.

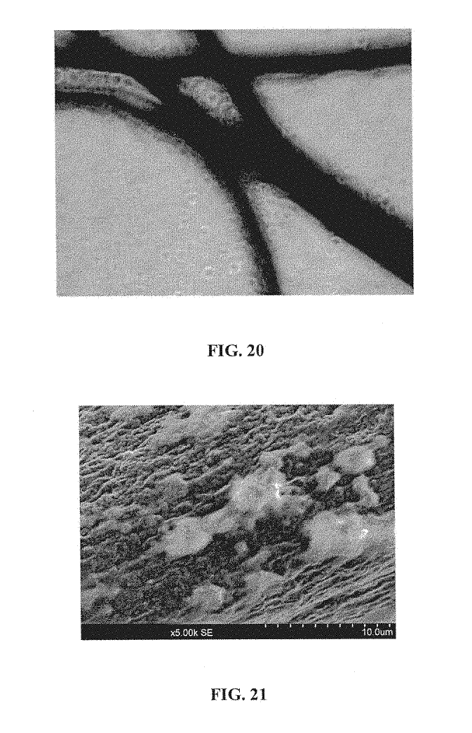

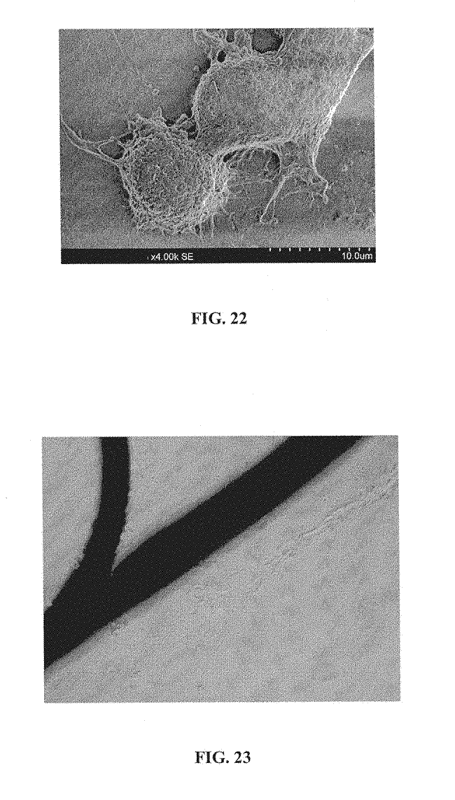

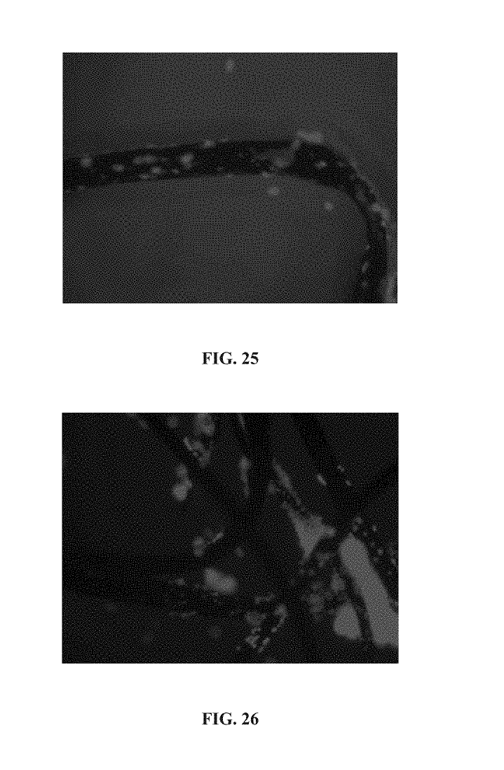

Cell adhesion was tested for the carbon nanotube-based fibers of the present specification using the previously prepared samples. For one series of samples, adhesive capacity was increased, using collagen coated plates, to avoid the agglomeration of cells. Another series of samples, comprising fibers that had first been treated with collagen by their immersion in a 0.5 mg/mL solution of sterilized Type II Collagen for 10 min, were assembled on 7×7 mm² glass slides. Alternatively, PLGA was also used. FIG. 18, FIG. 19, FIG. 20, FIG. 21 and FIG. 25 demonstrate cells adhesion to the various samples, displaying alignment of cells and the fibers. Adhesion was increased in presence of collagen and varied in function of the type of fiber.

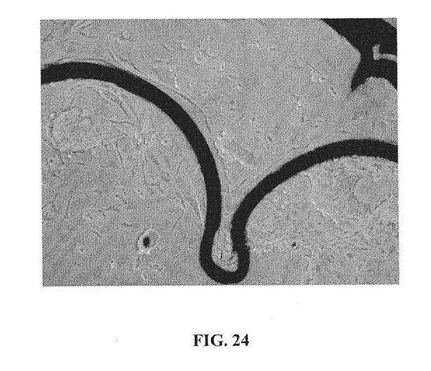

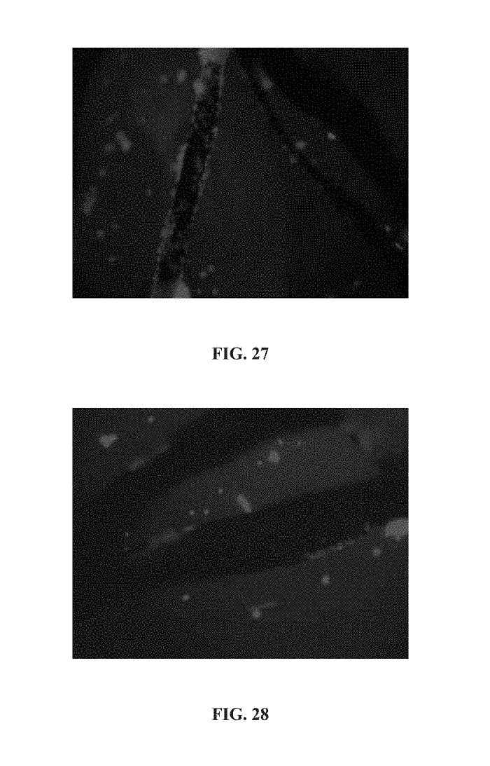

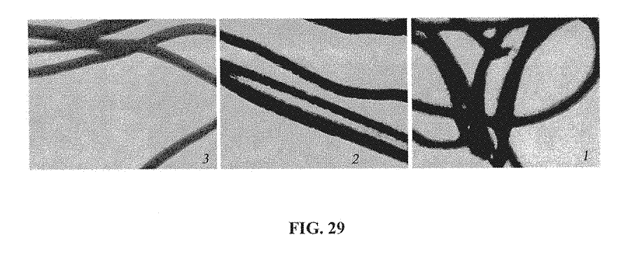

The PC12 cells were cultured in the presence/absence of neural growth factor (NGF). The cells were imaged using florescence microscopy to demonstrate the presence of cells on the fibers and their capacity to extend neurites in the presence of neural growth factors (NGFs). FIG. 23, FIG. 24, FIG. 25, FIG. 26, FIG. 27 and FIG. 28 demonstrate the formation of neurites.

The capacity of the carbon nanotube-based fibers of the present specification to encourage cell migration was evaluated by locally seeding PC12 cells, in 0.05 mL of culture media, on the sample. Visualization of the samples by light microscopy demonstrated the migration of cells as a function of the type of fiber. FIG. 29 demonstrates the migration of PC12 cells from an initial position where the cells were seeded, to an opposite side of the sample.

Human Skin Fibroblasts.

Human skin fibroblasts were used to investigate the ability of the carbon nanotube-based fibers of the present specification to form a fibrilar network of the extracellular matrix to sustain cell proliferation. These experiments demonstrate the potential use of the carbon nanotube-based fibers of the present specification as biomaterials for the attachment, alignment and proliferation of cells.

General Procedure for Human Skin Fibroblasts Bio-Assay.

Fibroblasts are generally known as anchorage-dependant cells. The carbon nanotube-based fibers described in Table 1 were used in the bio-assays. Pre-cleaned microscope glass slide (Erie Scientific, lot No. 2951) of 1 mm thickness were used. Human skin fibroblasts were obtained from Clonetics (San Diego, Calif., USA), and were used in the cell cultures. Fibroblast growth medium was composed of Dulbecco's Modified Eagle (DME) medium supplemented with 10% Fetal Bovine Serum (FBS) purchased from Gibco (Burlington, Ontario, Canada) and penicillin G purchased from Sigma-Aldrich Canada Ltd. (Oakville, Ontario, Canada). Phosphate Buffered Saline (PBS) was purchased from Sigma-Aldrich Canada Ltd. (Oakville, Ontario, Canada).

General Procedure for Sample Preparation.

The procedure for sample preparation was similar to the one described for PC12 cells. The microscope slides were cut into pieces (7 mm×7 mm) using a diamond cutter. The cut slides were then cleaned in an ultrasonic bath, 20 minutes in acetone, followed by 20 minutes in distilled water. The fiber sample, comprising 20 carbon nanotube-based fibers, was supported on the 7×7 mm² glass slide. The control sample was a 7×7 mm² glass slide coated with a R2-1 film and a PVA film. The samples were placed in a 12-well tissue culture plate, which was sterilized with ethylene oxide (EO) gas at 37° C. following standard procedures, and washed once with phosphate buffered saline (PBS). Human skin fibroblasts were seeded in 12-well plates, density of 2.5×10⁴ cells/cm², and cultured for 3 days at 37° C., 90% humidity and under a CO₂ atmosphere.

General Procedure for MTT Assay.

The procedure was the same as the one described for PC12 cells. As shown in Table 4, the absorbance value (Optical Density, OD) varied as a function of the carbon nanotube-based fiber. The MTT values demonstrate the capacity of the single-wall carbon nanotube-based fibers and multi-wall carbon nanotube-based fibers of the present specification to support cell proliferation. It is suggested that the proliferation of the fibroblasts was promoted by fibers comprising a higher percentage of copolymer (i.e. MWR2-2 and SWR3-2).

TABLE 4
Optical Density of the Synthesized Carbon Nanotube-Based
Fibers after MTT Assay with Human Skin Fibroblasts.

| | Optical Density |
|---|---|
| Fiber | (OD) |
| Positive Control - 1 | 0.329 |
| Glass piece coated with R2-1 film | |
| Positive Control - 2 | 0.339 |
| Glass piece coated with PVA film | |
| SW-Ctrl. | 0.085 |
| SWR2-1 | 0.085 |
| SWR3-1 | 0.083 |
| SWR2-2 | 0.073 |
| SWR3-2 | 0.102 |
| MW-Ctrl. | 0.095 |
| MWR2-1 | 0.100 |
| MWR3-1 | 0.089 |
| MWR2-2 | 0.255 |
| MWR3-2 | 0.092 |

General Procedure for the Observation of Cultured Human Skin Fibroblasts.

The cultured fibroblasts were observed using the same techniques and procedures as described for PC12 cells (i.e. inverted microscopy, scanning electron microscopy and Hoechst staining).

General Procedure for Human Skin Fibroblast Adhesion and Growth.

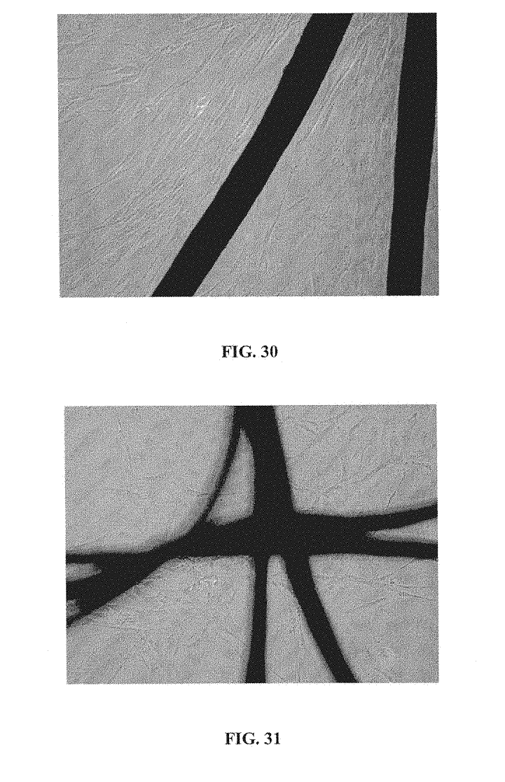

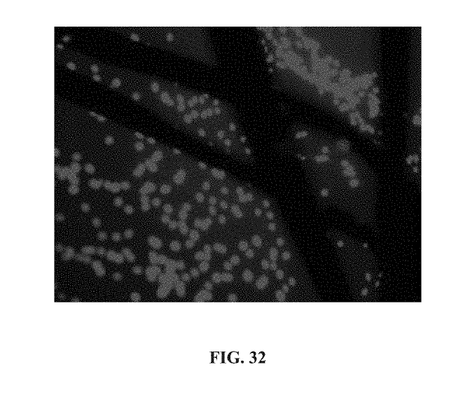

Cell adhesion was tested for the carbon nanotube-based fibers of the present specification using the previously prepared samples; no collagen or other protein coating was used. Inverted microscopy images of cultured cells demonstrated their adhesion to the fibers and their growth along the fibers, as illustrated in FIG. 30 and FIG. 31. Florescence microscopy demonstrated the cells uniformly spreading along fibers after 3 days, as illustrated in FIG. 32.

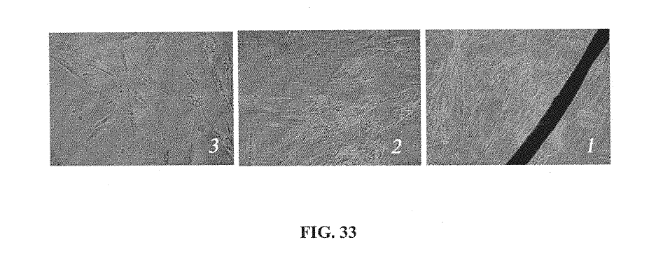

The capacity of the carbon nanotube-based fibers of the present specification to encourage cell migration was evaluated by locally seeding human skin fibroblasts. Visualization of the samples by light microscopy demonstrated the migration of the cells in function of the type of fiber. FIG. 33 demonstrates the migration of human skin fibroblasts from an initial position to an opposite side of the sample.

It is to be understood that the present disclosure is not limited in its application to the details of construction and parts as described hereinabove. The disclosure is capable of other embodiments and of being practiced in various ways. It is also understood that the phraseology or terminology used herein is for the purpose of description and not limitation. Hence, although the present disclosure has been described hereinabove by way of illustrative embodiments thereof, it can be modified, without departing from the spirit, scope and nature of the subject disclosure as defined in the appended claims.

### REFERENCES

[1] a) M. S. Dresselhaus, G. Dresselhaus, R. Saito, Carbon 1995, 33, 883; b) S. Iijima, Physica B 2002, 323, 1.
[2] O. Zhou, H. Shimoda, B. Gao, S. Oh, L. Fleming, G. Yue, Materials Science of Carbon Nanotubes: Fabrication, Integration, and Properties of Macroscopic Structures of Carbon Nanotubes, Acc. Chem. Res. 2002, 35, 1045.
[3] R. Sainz, A. M. Benito, M. T. Martinez, J. F. Galindo, J. Sotres, A. M. Baro, B. Corraze, O. Chauvet, A. B. Dalton, R. H. Baughman, W. K. Maser, A Soluble and Highly Functional Polyaniline-Carbon Nanotube Composite, Nanotechnology 2005, 16, S150.
[4] L. Y. Yeo, J. R. Friend, Electrospinning Carbon Nanotube Polymer Composite Nanofibers, Journal of Experimental Nanoscience 2006, 1, (2), 177.
[5] H. Paloniemi, M. Lukkarinen, T. Aaritalo, S. Arteva, J. Leiro, M. Heinonen, H. Haapakka, J. Lukkari, Layer-by-Layer Electrostatic Self-Assembly of Single-Wall Carbon Nanotube Polyelectrolytes, Langmuir 2006, 22, 74.
[6] S. E. Baker, K.-Y. Tse, E. Hindin, B. M. Nichols, T. L. Clare, R. J. Hamers, Chem. Mater. 2005, 17, (20), 4971.
[7] M. P. Mattson, R. C. Haddon, A. M. Rao, J. Molec. Neurosc. 2000, 14, (175), 175.
[8] J. L. McKenzie, R. Shi, B. E. Cardona, T. J. Webster AICh 2002.
[9] J. L. McKenzie, M. C. Waid, R. Shi, T. J. Webster Biomaterials 2004, 25, 1309.
[10] H. Hu, Y. Ni, V. Montana, R. Haddon, V. Parpura, Chemically Functionalized Carbon Nanotubes as Substrates for Neuronal Growth, Nano Letters 2004, 4, (3), 507.
[11] V. Lovat, D. Pantarotto, L. Lagostena, B. Cacciari, M. Grandolfo, M. Righi, G. Spalluto, M. Prato and L. Ballerini, Carbon Nanotube Substrates Boost Neuronal Electric Signaling, Nano Letters 2005, 5, (6), 1107.
[12] M. K. Gheith, V. A. Sinani, J. P. Wicksted, R. L. Matts, N. A. Kotov, Single-Walled Carbon Nanotube Polyelectrolyte Multilayers and Freestanding Films as a Biocompatible Platform for Neuroprosthetic Implants, Advanced Materials 2005, 17, 2663.
[13] H. Hu, Y. Ni, S. K. Mandal, V. Montana, B. Zhao, R. C. Hadon, V. Parpura, Polyethhylimine Functionalized Single-Walled Carbon Nanotubes as a Substrate for Neuronal Growth, The Journal of Physical Chemistry B 2005, 109, 4285.
[14] T. J. Webster, M. C. Waid, J. L. McKenzie, R. L. Price, J. U. Ejiofor, Nano-Biotechnology: Carbon Nanofibers as Improved Neural and Orthopaedic Implants, Nanotechnology 2004, 15, 48.
[15] R. L. Price, K. M. Haberstroh, T. J. Webster, Improved Osteoblast Viability in the Presence of Smaller Nanometer Dimensioned Carbon Fibers, Nanotechnology 2004, 15, 892.
[16] R. L. Price, M. C. Waid, K. M. Haberstroh, T. J. Webster, Selective Bone Cell Adhesion on Formulations Containing Carbon Nanofibers, Biomaterial 2003, 24, 1877.
[17] B. Zhao, H. Hu, S. K. Mandal, R. C. Haddon, A Bone Mimic Based on the Self-Assembly of Hydroxyapatite on Chemically Functionalized Single-Walled Carbon Nanotubes, Chem. Mater. 2005, 17, 3235.
[18] L. P. Zanello, B. Zhao, H. Hu, R. C. Haddon, Bone Cell Proliferation on Carbon Nanotubes, Nano Letter 2006, 6, (3), 562.
[19] D. J. Bryan, J. B. Tang, S. A. Doherty, D. D. Hile, D. J. Trantolo, D. L. Wise, I. C. Summerhayes, Enhanced Peripheral Nerve Regeneration through a Poled Bioasorbable Poly(lactic-co-glycolic acid) Guidance Channel, J. Neur. Eng. 2004, 1, 91.
[20] a) B. Vigolo, A. Pénicaud, C. Coulon, C. Sauder, R. Pailler, C. Journet, P. Bernier, P. Poulin, Macroscopic Fibers and Ribbons of Oriented Carbon Nanotubes, Science 2000, 290, 1331; b) P. Poulin, B. Vigolo, P. Launois, Film and Fibers of Oriented Single Wall Nanotubes, Carbon 2002, 40, 1741; c) International Application PCT/FR02/02804 published on Feb. 20, 2003 under WO 03/014431; d) International Application PCT/FR04/001034 published on Nov. 18, 2004 under WO 04/099475.
[21] R. H. Baughman, Putting a New Spin on Carbon Nanotubes, Science 2000, 290, pp. 1310.

1-52. (canceled)

53. A biocompatible carbon nanotube-based fiber comprising:
a) at least one carbon nanotube;
b) a nanoparticle of a biodegradable copolymer; and
c) a coagulating polymer matrix,
wherein the biocompatible carbon nanotube-based fiber comprises from about 15% to about 50% of the carbon nanotubes and from about 50% to about 85% of polymer, and
wherein the at least one carbon nanotube and the nanoparticle of a biodegradable copolymer comprise a binary colloidal mixture dispersed as a fiber within said matrix and wherein the surface of said fiber is capable of stimulating and sustaining cell proliferation.

54. The biocompatible carbon nanotube-based fiber of claim 53, wherein said carbon nanotube comprises a single-wall carbon nanotube.

55. The biocompatible carbon nanotube-based fiber of claim 53, wherein said carbon nanotube comprises a multi-wall carbon nanotube.

56. The biocompatible carbon nanotube-based fiber of claim 53, wherein said biodegradable copolymer is selected from the group consisting of polylactic-co-glycolic acid, polylactide-block-polyethylene oxide, polylactide-co-polycaprolactone, ethylene-co-vinyl alcohol and mixtures thereof.

57. The biocompatible carbon nanotube-based fiber of claim 56, wherein said biodegradable copolymer is polylactic-co-glycolic acid.

58. The biocompatible carbon nanotube-based fiber of claim 53, wherein said coagulating polymer is selected from the group consisting of polyvinyl alcohol, carboxymethyl cellulose, sodium alginate, hyaluronic acid and mixtures thereof.

59. The biocompatible carbon nanotube-based fiber of claim 58, wherein said coagulating polymer is polyvinyl alcohol.

60. The biocompatible carbon nanotube-based fiber of claim 53, further comprising additives selected from the group consisting of antibodies, chemical entities, collagen, drugs, growth factors, laminine, oligonucleotides, peptides, peptide derivatives, siRNA, and mixtures thereof.
